(19) United States
(12) Patent Application Publication
(10) Pub. No.: US 2019/0321239 A1
(43) Pub. Date: Oct. 24, 2019

(54) METHOD AND APPARATUS RELATED TO WIRELESSLY DETECTABLE SPONGES HAVING A WIRELESS TRANSPONDER

(71) Applicant: Covidien LP, Mansfield, MA (US)

(72) Inventors: Allan Aquino, Longmont, CO (US); Kim Brandt, Loveland, CO (US); Andy Buersmeyer, Ft. Collins, CO (US)

(21) Appl. No.: 16/351,630

(22) Filed: Mar. 13, 2019

Related U.S. Application Data
(60) Provisional application No. 62/661,144, filed on Apr. 23, 2018.

(51) Int. Cl. A61F 13/44; H04B 1/02; A61F 13/15; A61F 13/36; C09J 5/00

(52) U.S. Cl. CPC A61F 13/44; H04B 1/02; A61F 13/15747; A61F 2013/8479; A61F 13/15804; C09J 5/00; A61F 2013/1591; A61F 13/36

(57) ABSTRACT

A wirelessly detectable sponge and process for making same are disclosed. The wirelessly detectable sponge may include a sponge, a first deposit of adhesive, a wireless transponder, and a second deposit of adhesive. The first and second deposits of adhesive may be the same or different type of adhesive. The adhesive may be deposited using one or more automated dispensers that may be arranged relative to an assembly system that moves the sponges along a conveyor, or relative to an assembly system in which sponges are processed along a stationary surface. The wireless transponders may be picked by a robotic appendage, moved relative to the sponge, and placed within a defined location at a defined orientation.

### CROSS-REFERENCE TO RELATED APPLICATIONS

This application claims the benefit of and priority to U.S. Provisional Patent Application Ser. No. 62/661,144 filed Apr. 23, 2018, the entire disclosure of which is incorporated by reference herein.

### BACKGROUND

### Technical Field

The present disclosure generally relates to physically coupling a wireless transponder to a sponge using deposits of adhesive that are deposited onto the sponge.

### Description of the Related Art

It is important to determine whether objects or items associated with a medical or clinical procedure are present or unintentionally retained in a patient's body before completion of a medical or clinical procedure. The medical or clinical procedure may, for example, take the form of a surgery or childbirth delivery. Such objects or items may take a variety of forms used in medical or clinical procedures, and may include instruments (e.g., scalpels, scissors, forceps, hemostats, and/or clamps) that may be reusable after sterilization, or alternatively single-use disposable accessories, objects and/or items (e.g., disposable surgical sponges, gauzes, and/or absorbent pads). When used in surgery, failure to locate an object or item before closing the patient may require additional surgery, and in some instances may have serious adverse medical consequences. In other medical procedures, such as vaginal childbirth deliveries, failure to remove objects, for instance gauze or absorbent pads, can lead to infections and undesired complications.

Some hospitals have instituted procedures that include checklists or requiring multiple manual counts to be performed to track the use and return of objects or items during surgery. Such a manual approach is inefficient, requiring the time of highly trained personnel, and is prone to error.

Another approach employs wireless transponders that are attached to various objects or items used during surgery, and a wireless interrogation and detection system. Such an approach can employ “dumb” wireless transponders, i.e., wireless communications transponders that do not store and/or transmit any unique identifying information. Dumb wireless transponders have traditionally been employed for electronic article surveillance (EAS) to prevent loss of merchandise at retail locations. Alternatively, such an approach can employ radio frequency identification (RFID) wireless transponders, i.e., wireless communications transponders which do store and return a unique identifier in response to an interrogation signal emitted by an RFID interrogator or RFID reader.

In an approach that employs RFID wireless transponders, an interrogator or reader includes a transmitter that emits wireless interrogation signals (e.g., radio or microwave frequency) and a detector for detecting wireless response signals returned by the RFID wireless transponders in response to the emitted interrogation signals. Such an automated system advantageously detects the unique identifiers of the RFID wireless transponders; however since some of the power in the interrogation signal is required to operate the RFID wireless transponder such an approach may have shorter range or less ability to detect objects or items retained within bodily tissue as compared to dumb wireless transponders communicating in similar ranges of wavelength and levels of power.

Commercial implementation of such an automated system requires that the overall system be cost competitive, highly accurate, and easy to use. In particular, false negatives must be avoided to ensure that objects are not mistakenly left in the patient and false positives avoided to ensure valuable time and resources are not spent looking for objects which were not actually retained in the patient. In addition, the wireless transponders should be detectable by the RFID interrogator and/or reader. To improve detectability, the wireless transponder may be located at a consistent location and/or orientation for each item that carries a wireless transponder. Currently implemented manufacturing methods for attaching wireless transponders to disposable items or accessories, such as sponges, however, are labor intensive and result in uncertainty regarding the location and/or orientation of the wireless transponder. Moreover, in some implementations, the wireless transponder is contained within, and free to move about, a pouch that is physically coupled to the disposable item or accessory.

### BRIEF SUMMARY

Automating the placement and attachment of the wireless transponder on a type of sponge may advantageously provide consistency in positioning and orienting the wireless transponder when manufacturing the sponge type.

A process to manufacture wirelessly detectable sponges for use in medical procedures may be summarized as including: depositing a first deposit of adhesive onto a first sponge at a first defined location on the first sponge; picking a first wireless transponder by a robotic appendage from a supply of wireless transponders; moving the robotic appendage to position the picked first wireless transponder at least proximate the first defined location on the first sponge; placing the picked first wireless transponder onto the first deposit of adhesive at the first defined location on the first sponge; depositing a second deposit of adhesive to cover the wireless transponder; and curing at least one of the first or the second deposits of adhesive.

The first sponge may have a length, a width and a thickness, and placing the picked first wireless transponder onto the first deposit of adhesive at the first defined location on the first sponge may include placing the picked first wireless transponder onto the first deposit of adhesive at the first defined location on the first sponge in a defined orientation relative to the length and width of the first sponge. Depositing a second deposit of adhesive to cover the wireless transponder may include depositing the second deposit of adhesive to enclose the wireless transponder by a combination of the first and the second deposits of adhesive. The robotic appendage may be a robotic arm that includes an end-of-arm tool, and picking a first wireless transponder by a robotic appendage may include moving the robotic arm to position the end-of-arm component with respect to a storage container that holds the supply of wireless transponders. Depositing a first deposit of adhesive onto a first sponge at a first defined location on the first sponge may include depositing the first deposit of adhesive onto the first sponge at the first defined location on the first sponge via a first automated dispenser. The automated dispenser may be carried by a robotic appendage and depositing the first deposit of adhesive onto the first sponge at the first defined location on the first sponge via an automated dispenser may include positioning the automated dispenser relative to the first sponge by the robotic appendage. Depositing a second deposit of adhesive to cover the wireless transponder may include depositing the second deposit of adhesive via the first automated dispenser. Depositing a second deposit of adhesive to cover the wireless transponder may include depositing the second deposit of adhesive via a second automated dispenser. Depositing a first deposit of adhesive onto a first sponge at a first defined location on the first sponge may include depositing a first amount of a first type of adhesive onto the first sponge at the first defined location on the first sponge, and depositing a second deposit of adhesive to cover the wireless transponder may include depositing a second amount of the first type of adhesive to cover the wireless transponder. The process may further include: folding a portion of the first sponge to cover the first transponder before depositing the second deposit of adhesive to cover the wireless transponder. Curing at least one of the first or the second deposits of adhesive may include illuminating at least one of the first or the second deposits of adhesive with ultraviolet light for a period of time.

A wirelessly detectable sponge may be summarized as including: a first sponge; a first deposit of adhesive, the first deposit of adhesive which is deposited onto the first sponge at a first defined location; a first wireless transponder, the first wireless transponder which is positioned on the first deposit of adhesive at a first defined location on the first sponge; and a second deposit of adhesive, the second deposit of adhesive which is deposited to cover the first wireless transponder.

The first sponge may have a length, a width and a thickness, and the first wireless transponder may be positioned onto the first deposit of adhesive at the first defined location in a defined orientation relative to the length and width of the first sponge. The first wireless transponder may be enclosed by a combination of the first deposit of adhesive and the second deposit of adhesive. The first deposit of adhesive may include a first amount of a first type of adhesive, and the second deposit of adhesive may include a second amount of the first type of adhesive. A portion of the first sponge may be folded to be between the first transponder and the second deposit of adhesive to cover the wireless transponder.

An assembly system that produces wirelessly detectable sponges may be summarized as including: an assembly surface, the assembly surface which supports a first sponge; a robotic appendage, the robotic appendage which picks a first wireless transponder from a supply of wireless transponders, and places the first wireless transponder onto the first sponge at a defined location; at least one automated dispenser, the at least one automated dispenser which dispenses a first deposit of adhesive onto the first sponge at the defined location during a first time period before the first wireless transponder is placed onto the first sponge, and the at least one automated dispenser which dispenses a second deposit of adhesive onto the first sponge at the defined location during a second time period after the first wireless transponder is placed onto the sponge; and an illumination source, the illumination source which is positioned to illuminate at least one of the first deposit of adhesive and the second deposit of adhesive with ultraviolet light.

The assembly surface may be comprised of a conveyor. The at least one automated dispenser may include a first automated dispenser that dispenses the first deposit of adhesive during the first time period and that dispenses the second deposit of adhesive during the second time period. The at least one automated dispenser may include a first automated dispenser that dispenses the first deposit of adhesive during the first time period and a second automated dispenser that dispenses the second deposit of adhesive during the second time period. The robotic appendage may be a robotic arm that includes an end-of-arm tool, and picking the first wireless transponder by the robotic appendage may include moving the robotic arm to position the end-of-arm tool with respect to a storage container that holds the supply of wireless transponders.

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings.

FIG. 1A is an isometric view of a wirelessly detectable sponge in which two deposits of adhesive secure a wireless transponder to a sponge, according to at least one illustrated implementation.

FIG. 1B is a side elevation view of the two deposits of adhesive and the wireless transponder shown in FIG. 1A.

FIG. 2A is an isometric view of a wirelessly detectable sponge in which a portion of a sponge has been folded between two deposits of adhesive secure that secure a wireless transponder to the sponge, according to at least one illustrated implementation.

FIG. 2B is a side elevation view of the two deposits of adhesive, the folded sponge, and the wireless transponder shown in FIG. 2A.

FIG. 3A is a top plan view of an assembly system that includes an assembly surface for producing wireless detectable sponges, in which the assembly system includes at least one automated dispenser that dispenses adhesive onto the sponges, and at least one robotic appendage that places wireless transponders onto the sponges at a defined location, according to at least one illustrated implementation.

FIG. 3B is a top plan view of an assembly system that includes a processing station that may be used to produce wireless detectable sponges, in which the processing station includes one or more automated dispensers that dispense adhesive, and at least one robotic appendage that picks and places wireless transponders onto the sponges at a defined location, according to at least one illustrated implementation.

FIG. 4 is an isometric view of a dispenser that may dispense adhesive, according to at least one illustrated implementation.

FIG. 5A is an isometric view of a robotic appendage that picks and places wireless transponders from a supply of wireless transponders that are delivered via a tape and reel, according to at least one illustrated implementation.

FIG. 5B is an isometric view of a robotic appendage that picks and places wireless transponders from a supply of wireless transponders contained within a storage container, according to at least one illustrated implementation.

FIG. 6 is a flow diagram showing a process of manufacturing wireless detectable sponges, according to at least one illustrated implementation.

FIG. 7 is a flow diagram showing a process for folding a portion of the sponge as part of manufacturing a wireless detectable sponge, according to at least one illustrated implementation.

### DETAILED DESCRIPTION

In the following description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures associated with transmitters, receivers, or transceivers and/or medical equipment and medical facilities have not been shown or described in detail to avoid unnecessarily obscuring descriptions of the embodiments.

Unless the context requires otherwise, throughout the specification and claims which follow, the word “comprise” and variations thereof, such as “comprises” and “comprising,” are to be construed in an open, inclusive sense, that is as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The headings and Abstract of the Disclosure provided herein are for convenience only and do not interpret the scope or meaning of the embodiments.

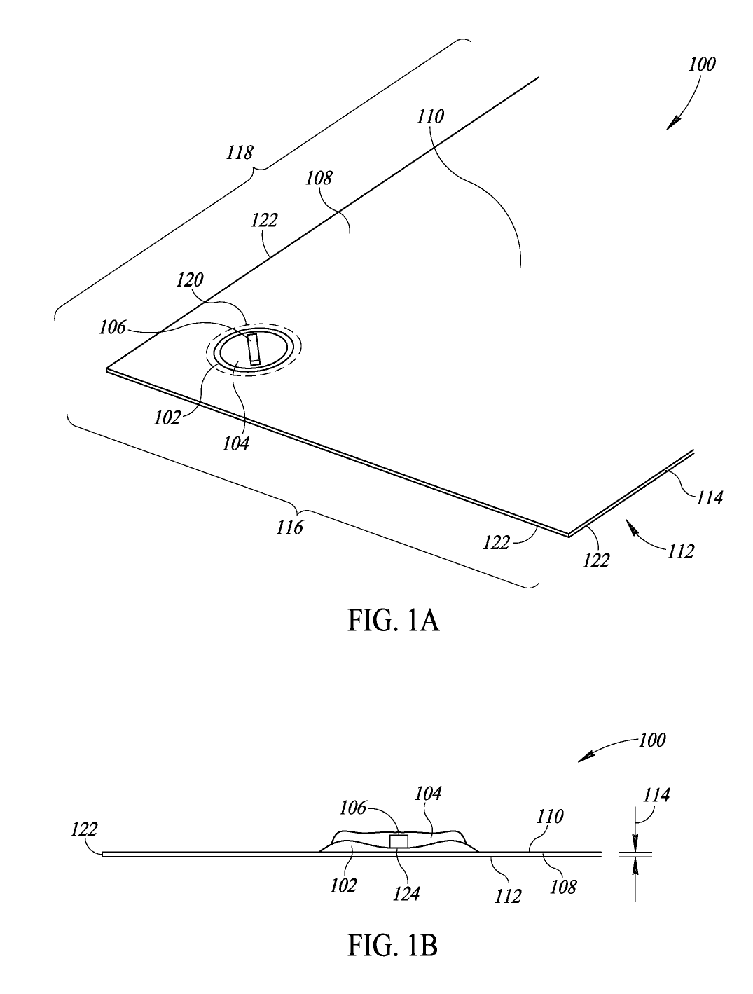

FIGS. 1A and 1B show a wirelessly detectable sponge 100 in which a first deposit of adhesive 102 and a second deposit of adhesive 104 secure a wireless transponder 106 to a sponge 108, according to at least one illustrated implementation. The sponge 108 may include a first primary surface 110 and a second, opposing primary surface 112 separated by a thickness 114. The sponge 108 may have a length 116 and a width 118 formed by the first primary surface 110. The first deposit of adhesive 102 may be made onto the first primary surface 110 of the sponge 108. In some implementations, the first deposit of adhesive 102 may be within a defined location 120 along the first primary surface 110, such as, for example, along one or more edges 122 (such as, e.g., proximate one corner) of the sponge 108. In a manufacturing setting in which a plurality of sponges are processed, such a defined location 120 for depositing the first adhesive 102 may remain constant for each of the plurality of sponges 108 being processed. The sponge 108 may include disposable, single use absorbent surgical sponges, gauze and/or padding suitable for use in an operating room environment.

The first deposit of adhesive 102 may include one or more types of adhesive that are approved for use in a surgical environment. In some implementations, the first deposit of adhesive 102 may be biocompatible, permitting use in vivo. The first deposit of adhesive 102 may, for example, take the form of an adhesive web film. The first deposit of adhesive 102 may, for example, take the form of a thermal lamination film. The first deposit of adhesive 102 may, for example, take the form of a meltable plastic layer, such as, for example, a thermoplastic layer. The first deposit of adhesive 102 may be a thermosetting plastic that has an initial cure temperature at which the thermosetting plastic layer cures, such as, for example, a temperature less than 130 degrees Centigrade. The first deposit of adhesive 102 may be a heat-activated adhesive layer. Alternatively or additionally, the first deposit of adhesive 102 may be pressure-activated adhesive layer or a pressure-sensitive adhesive layer. Alternatively or additionally, the first deposit of adhesive 102 may be a water-activated adhesive. Alternatively or additionally, the first deposit of adhesive 102 may be a radiation curing adhesive that may be cured by illuminating the first deposit of adhesive 102 with one or more types of radiation (e.g., ultraviolet (“UV”) light, and visible light) for a period of time. The first deposit of adhesive 102 may include, for example, acrylate resins (e.g., acrylate epoxies, polyurethanes, and silicones), photoinitiators, photosensitizers, and/or monomers.

The wireless transponder 106 may be selectively placed onto the first deposit of adhesive 102 before the first deposit of adhesive 102 is cured or set. As a result, the wireless transponder 106 may make a depression or groove 124 within the first deposit of adhesive 102 when the wireless transponder 106 is placed onto the first deposit of adhesive 102. When the first deposit of adhesive 102 is cured, the depression or groove 124 may be used to form a portion of an encapsulation that surrounds the wireless transponder 106. In some implementations, the wireless transponder 106 may be oriented along the sponge 108 in a defined direction. For example, the wireless transponder 106 may be placed onto the first deposit of adhesive 102 at the defined location 120 on the sponge 108 in a defined orientation relative to the length 116 and/or width 118 of the sponge 108. For example, in some implementations in which the wireless transponder 106 is located proximate an edge 122 of the sponge 108, the wireless transponder 106 may be oriented to extend parallel to the edge 122 of the sponge 108. In some implementations in which the wireless transponder 106 is located proximate a corner formed by two intersecting edges 122 of the sponge, the wireless transponder 106 may be oriented to extend perpendicular to a line that bisects the angle formed by the two intersecting edges of the sponge.

The wireless transponder 106 may be comprised of one or more RFID transponders or RFID tags that encode unique identifiers that may uniquely identify the wireless transponder 106. The RFID transponders or RFID tags store and return the unique identifiers (e.g., unique at least within a large enough set to supply a large clinical facility for a month). The RFID transponders or RFID tags may, preferably, take the form of passive RFID transponders or RFID tags which omit batteries and derive power for operation from the interrogation signal. While denominated as “radio frequency,” the RFID transponders or tags typically may operate or communicate in the low or high frequency (e.g., radio frequency) and/or ultra-high frequency (e.g., microwave frequency) portions of the electromagnetic spectrum. Hence, consistent with common usage in the field of automatic data collection, use of the terms radio frequency and/or RFID is not limited to interrogation systems and wireless communications transponders that employ radio frequency communications, but also include interrogation systems and wireless communications transponders that employ microwave frequency communications.

The wireless transponder 106 may be comprised of one or more dumb transponders that do not encode unique identifiers, but may return a signal that may be used determine at least one of a presence or absence of the wireless communications dumb transponders in the range of a wireless communications presence/absence interrogation system(s). The wireless communications dumb transponders may be simple LC resonant circuits that do not store, encode or return unique identifiers. The wireless communications dumb transponders may communicate within a lower frequency range than RFID transponders or RFID tags. The communication at a relatively lower frequency may advantageously result in better range than obtainable by the RFID transponders or RFID tags, and provide an increased ability to detect a wireless communications dumb transponder retained in bodily tissue, even where a patient is obese. In some instances, the frequency range of the RFID transponder and the wireless communications dumb transponder may not overlap.

The second deposit of adhesive 104 may be deposited within the defined location 120 onto at least a portion of the first deposit of adhesive 102. The second deposit of adhesive 104 may cover the wireless transponder 106. In such an implementation, the first deposit of adhesive 102 and the second deposit of adhesive 104 may collectively enclose the wireless transponder 106.

The second deposit of adhesive 104 may be the same type of adhesive as the first deposit of adhesive 102. Alternatively, the second deposit of adhesive 104 may be a different type of adhesive than the first deposit of adhesive 102. The second deposit of adhesive 104 may include one or more types of adhesive that are approved for use in a surgical environment. In some implementations, the second deposit of adhesive 104 may be biocompatible, permitting use in vivo. The second deposit of adhesive 104 may, for example, take the form of an adhesive web film. The second deposit of adhesive 104 may, for example, take the form of a thermal lamination film. The second deposit of adhesive 104 may, for example, take the form of a meltable plastic layer, such as, for example, a thermoplastic layer. The second deposit of adhesive 104 may be a thermosetting plastic that has an initial cure temperature at which the thermosetting plastic layer cures, such as, for example, a temperature less than 130 degrees Centigrade. The second deposit of adhesive 104 may be a heat-activated adhesive layer. Alternatively or additionally, the second deposit of adhesive 104 may be pressure-activated adhesive layer or a pressure-sensitive adhesive layer. Alternatively or additionally, the second deposit of adhesive 104 may be a water-activated adhesive. Alternatively or additionally, the second deposit of adhesive 104 may be a radiation curing adhesive that may be cured via illuminating the second deposit of adhesive with one or more types of radiation (e.g., ultraviolet (“UV”) light, and visible light) for a period of time. The second deposit of adhesive 104 may include, for example, acrylate resins (e.g., acrylate epoxies, polyurethanes, and silicones), photoinitiators, photosensitizers, and/or monomers.

In some implementations, the second deposit of adhesive 104 may be deposited onto the first deposit of adhesive 102 before the first deposit of adhesive 102 is cured. In such an implementation, the first deposit of adhesive 102 and the second deposit of adhesive 104 may be cured (e.g., through exposure to a specified temperature, and/or exposure to radiation of a defined wavelength) at the same time. In some implementations, the first deposit of adhesive 102 may be cured before the second deposit of adhesive 104 is placed onto the first deposit of adhesive 102. The second deposit of adhesive 104 may thereby be cured separately from the first deposit of adhesive 102. When cured, the combination of the first deposit of adhesive 102 and the second deposit of adhesive 104 may encapsulate the wireless transponder 106.

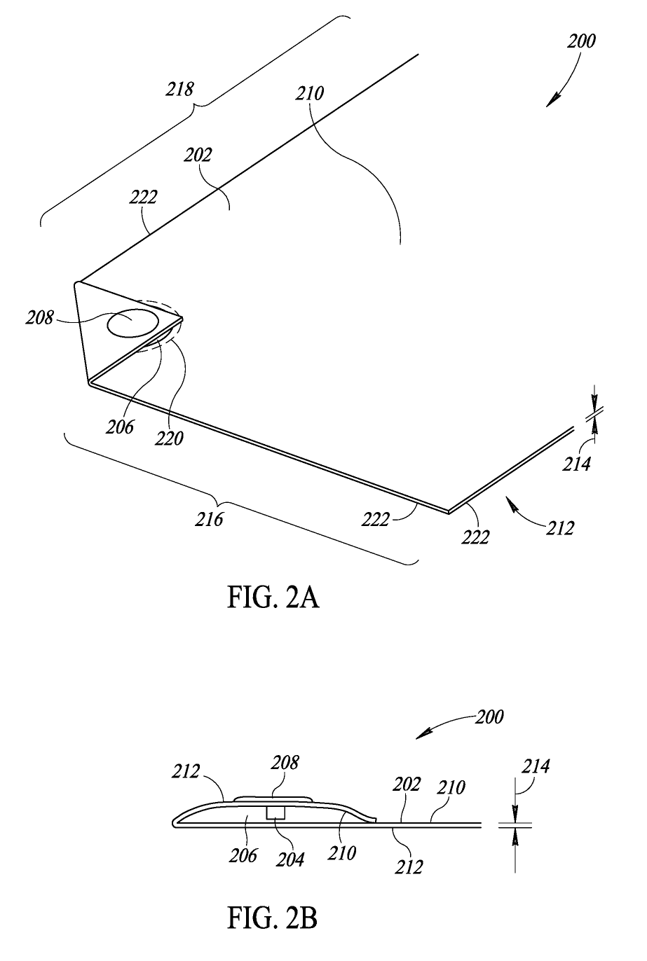

FIGS. 2A and 2B show a wirelessly detectable sponge 200 in which a portion of a sponge 202 has been folded over a wireless transponder 204 and between a first deposit of adhesive 206 and a second deposit of adhesive 208 that secure the wireless transponder 204 to the sponge 202, according to at least one illustrated implementation. The sponge 202 may include disposable, single use absorbent surgical sponges, gauze and/or padding suitable for use in an operating room environment. The sponge 202 may include a first primary surface 210 and a second, opposing primary surface 212 separated by a thickness 214. The sponge 202 may have a length 216 and a width 218 formed by the first primary surface 210. The first deposit of adhesive 206 may be made onto the first primary surface 210 of the sponge 202. In some implementations, the first deposit of adhesive 102 may be within a defined location 220 along the first primary surface 210, such as, for example, along one or more edges 222 (such as, e.g., proximate one corner) of the sponge 202.

The wireless transponder 204 may be placed onto the first deposit of adhesive 206. A portion of the sponge 202 may then be folded over the defined location 220 to thereby cover the wireless transponder 204 and at least a portion of the first deposit of adhesive 206. As such, a portion of the second opposing primary surface 212 may thereby be exposed at the defined location 220. The second deposit of adhesive 208 may thereby be deposited onto the exposed portion of the second opposing primary surface 212 at the defined location 220 to thereby cover the wireless transponder 204. The wireless transponder 204 may be enclosed by a combination of the first deposit of adhesive 206, the second deposit of adhesive 208, and the portion of the sponge 202 folded over the defined location 220.

The first deposit of adhesive 206 may be cured (e.g., through exposure to a specified temperature, and/or exposure to radiation of a defined wavelength) before the sponge 202 is folded over the defined location 220. As such, the second deposit of adhesive 208 may be cured (e.g., through exposure to a specified temperature, and/or exposure to radiation of a defined wavelength) at a different time as the first deposit of adhesive 206 to thereby encapsulate the wireless transponder 204. In some implementations, the first deposit of adhesive 206 and the second deposit of adhesive 208 may be cured at the same time.

The first deposit of adhesive 206 may be the same as, or different than, the second deposit of adhesive 208. The first deposit of adhesive 206 and/or the second deposit of adhesive 208 may include one or more types of adhesive that are approved for use in a surgical environment. In some implementations, for example, first deposit of adhesive 206 and/or the second deposit of adhesive 208 may be biocompatible, permitting use in vivo. The first deposit of adhesive 206 and/or the second deposit of adhesive 208 may, for example, take the form of an adhesive web film. The first deposit of adhesive 206 and/or the second deposit of adhesive 208 may, for example, take the form of a thermal lamination film. The first deposit of adhesive 206 and/or the second deposit of adhesive 208 may, for example, take the form of a meltable plastic layer, such as, for example, a thermoplastic layer. The first deposit of adhesive 206 and/or the second deposit of adhesive 208 may be a thermosetting plastic that has an initial cure temperature at which the thermosetting plastic layer cures, such as, for example, a temperature less than 130 degrees Centigrade. The first deposit of adhesive 206 and/or the second deposit of adhesive 208 may be a heat-activated adhesive layer. Alternatively or additionally, the first deposit of adhesive 206 and/or the second deposit of adhesive 208 may be pressure-activated adhesive layer or a pressure-sensitive adhesive layer. Alternatively or additionally, the first deposit of adhesive 206 and/or the second deposit of adhesive 208 may be a water-activated adhesive. Alternatively or additionally, the first deposit of adhesive 206 and/or the second deposit of adhesive 208 may be a radiation curing adhesive that may be cured via illuminating the second deposit of adhesive with one or more types of radiation (e.g., ultraviolet (“UV”) light, and visible light) for a period of time. The first deposit of adhesive 206 and/or the second deposit of adhesive 208 may include, for example, acrylate resins (e.g., acrylate epoxies, polyurethanes, and silicones), photoinitiators, photosensitizers, and/or monomers.

The wireless transponder 204 may be comprised of one or more RFID transponders or RFID tags that encode unique identifiers that may uniquely identify the wireless transponder 204. Alternatively, or in addition, the wireless transponder 204 may be comprised of one or more dumb transponders that do not encode unique identifiers, but may return a signal that may be used determine at least one of a presence or absence of the wireless communications dumb transponders in the range of a wireless communications presence/absence interrogation system(s).

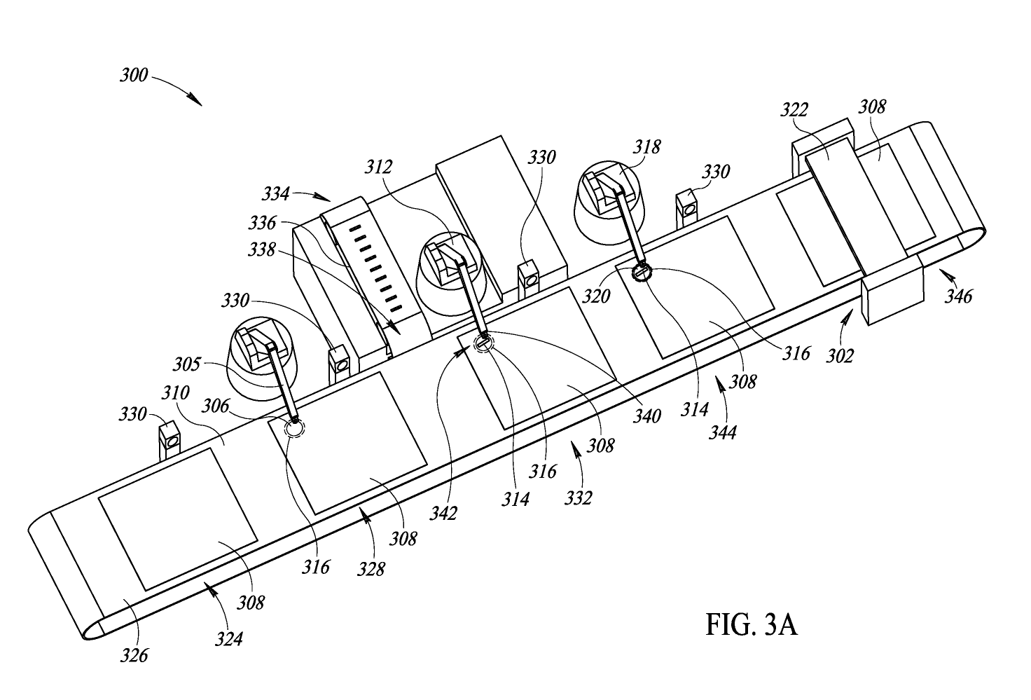

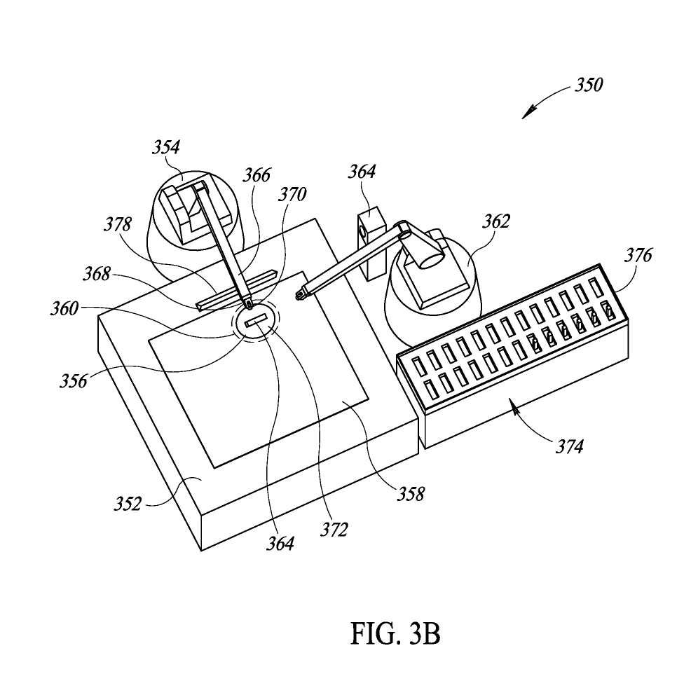

FIG. 3 shows an assembly system 300 that produces wireless detectable sponges 302, in which the assembly system 300 includes a first automated dispenser 304 that dispenses a first amount of adhesive 306 onto sponges 308 conveyed along an assembly surface 310, a robotic appendage 312 that places wireless transponders 314 onto the conveyed sponges 308 at a defined location 316, a second automated dispenser 318 that dispenses a second amount of adhesive 320 onto the sponges 308 at the defined location 316, and a illumination source 322 that emits radiation that illuminates the first amount of adhesive 306 and/or the second amount of adhesive 320, according to at least one illustrated implementation.

In some implementations, the sponges 308 may be placed onto the assembly surface 310 manually. Alternatively, the sponges 308 may be placed onto the assembly surface 310 using an automated process, such as, for example, when the assembly system 300 may be included as part of a larger assembly or processing system. In some implementations, a continuous sheet of absorbent material may be conveyed along the assembly surface 310 to be processed by one or more of the first automated dispenser 304, the robotic appendage 312, the second automated dispenser 318, and the illumination source 322. The continuous sheet may then be cut into individual wireless detectable sponges 302.

In some implementations, the assembly system 300 may include one or more sensors 330 that may be used to detect a position and/or orientation of the sponges 308 on the assembly surface 310. Such sensors 330 may include, for example, image sensors (e.g., imagers, cameras) that capture one or more images of portions of the assembly surface 310 and/or emitter-receiver pairs that may be used to detect the presence or absence of sponges 308 at one or more positions, as well as the orientation of detected sponges 308, along the assembly surface 310.

In some implementations, a sponge 308 may be placed onto the assembly surface 310 at a first location 324. The sensor and/or image 330 may detect the placement of the sponge 308 on the assembly surface 310. The sponge 308 may then be conveyed, using, for example, a conveyor 326, to a second location 328 proximate the first automated dispenser 304. The first automated dispenser 304 may be physically coupled to a first appendage 305 that may be selectively movable with multiple degrees of freedom. When the sponge 308 is detected at the second location 328 (via, for example, one or more sensors 330), the first automated dispenser 304 may be positioned using the first appendage 305 relative to the sponge 308 located at the second location 328 to dispense a first amount of adhesive 306 onto the sponge 308 at the defined location 316. The defined location 316, for example, may be proximate one or more of the edges of the sponge 308, such as, for example, the edge that is located proximate the first automated dispenser 304.

The first amount of adhesive 306 may include one or more types of adhesive that are approved for use in a surgical environment. In some implementations, for example, the first amount of adhesive 306 may be biocompatible, permitting use in vivo. The first amount of adhesive 306 may, for example, take the form of an adhesive web film. The first amount of adhesive 306 may, for example, take the form of a thermal lamination film. The first amount of adhesive 306 may, for example, take the form of a meltable plastic layer, such as, for example, a thermoplastic layer. The first amount of adhesive 306 may be a thermosetting plastic that has an initial cure temperature at which the thermosetting plastic layer cures, such as, for example, a temperature less than 130 degrees Centigrade. The first amount of adhesive 306 may be a heat-activated adhesive layer. Alternatively or additionally, the first amount of adhesive 306 may be pressure-activated adhesive layer or a pressure-sensitive adhesive layer. Alternatively or additionally, the first amount of adhesive 306 may be a water-activated adhesive. Alternatively or additionally, the first amount of adhesive 306 may be a radiation curing adhesive that may be cured via illuminating the second deposit of adhesive with one or more types of radiation (e.g., ultraviolet (“UV”) light, and visible light) for a period of time. The first amount of adhesive 306 may include, for example, acrylate resins (e.g., acrylate epoxies, polyurethanes, and silicones), photoinitiators, photosensitizers, and/or monomers.

After the first amount of adhesive 306 is deposited, the sponge 308 may be conveyed to a third location 332 along the assembly surface 310 proximate the robotic appendage 312. The robotic appendage 312 may be used to pick wireless transponders 314 from a source 334 of wireless transponders, and place individual wireless transponders 314 onto the sponge 308 at the defined location 316. As shown in FIG. 3A, the source 334 of wireless transponders may include a tape-and-reel component 336 in which the wireless transponders are stored and transported using a tape that is rolled into a reel. The tape is then unwound from the reel to provide access to a wireless transponder 314. In some implementations, the tape may be unwound in a direction towards the assembly surface 310. In such an implementation, the wireless transponders 314 may be located proximate the assembly surface 310 at the third location 332 when the wireless transponders 314 are picked from the tape.

The robotic appendage 312 may be used to pick wireless transponders 314 from the tape-and-reel component 336, and place the picked wireless transponder 314 on the sponge 308 located at the third location 332. The robotic appendage 312 may be operable to place the picked wireless transponder 314 onto the first deposit of adhesive 306 within the defined location 316. The location and orientation of the sponge 308 at the third location 332 may be detected by one or more sensors 330 that are positioned to detect items at the third location 332. In some implementations, the robotic appendage 312 may be mechanically coupled to one or more appendages and/or pivot points such that the robotic appendage 312 may be selectively moveable with at least 6 degrees of freedom. In some implementations, the robotic appendage 312 may use an end-of-arm tool 340 to selectively pick the wireless transponder 314 at a first position 338 that may correspond with a portion of the tape in the tape-and-reel component 336 that is proximate the third location 332. The end-of-arm tool 340 may include a plurality of opposable, mechanical digits that may selectively be operable to move between a closed position that may be used to pick and/or hold wireless transponders 314 and an open position that may be used to deposit and/or place the wireless transponders 314. In some implementations, the end-of-arm tool 340 may include a resiliently conformable member that has an associated port. The port may be fluidly coupled to a source of low pressure (e.g., a vacuum) that may be used to create suction at the port. Once the robotic appendage 312 picks the wireless transponder 314, the tape-and-reel component 336 may advance so that the next wireless transponder 314 on the tape will be located at the first position 338.

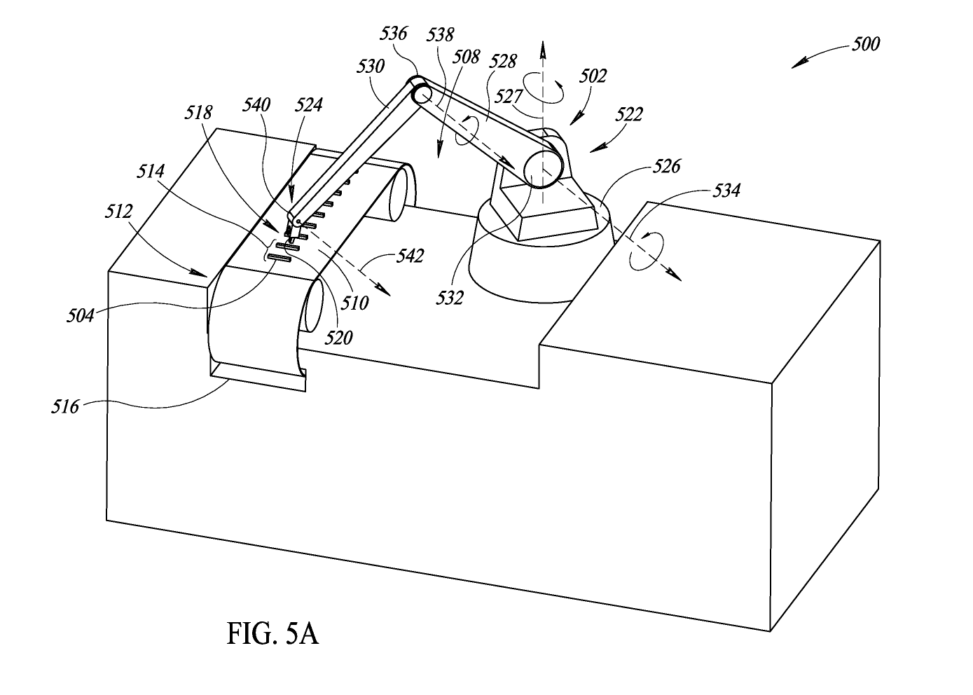

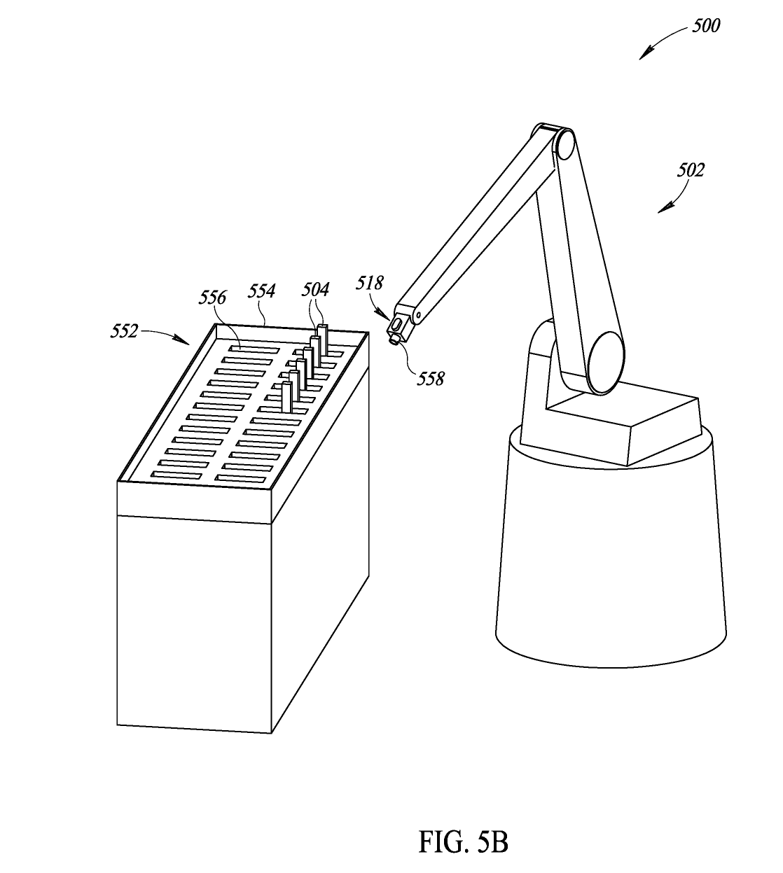

Once the robotic appendage 312 picks the wireless transponder 314, the robotic appendage 312 may move towards a second position 342 that may correspond or be related to the defined location 316 of the sponge 308 located at the third location 332 on the assembly surface 310. The robotic appendage 312 may then place the wireless transponder 314 onto the first deposit of adhesive 306 of the sponge 308 located at the third location 332. The robotic appendage 312 may then move back towards the first position 338 to prepare to pick the next wireless transponder 314 from the tape. In such an implementation, the robotic appendage 312 may be used to place multiple wireless transponders 314 onto the sponge 308. For example, in some implementations, the robotic appendage 312 may selectively pick different types of wireless transponders (e.g., an RFID transponder and a dumb wireless transponder), potentially from different sources 334 of wireless transponders (only one shown in FIG. 5). Once the robotic appendage 312 has completed processing the sponge 308 at the third location 332, the assembly surface 310 may then move the sponge 308 to a fourth location 344 proximate the second automated dispenser 318.

In some implementations, a space may be provided between that third location 332 and the fourth location 344 at which a portion of the sponge 308 may be selectively folded over the defined location 316. Such folding may occur manually, and/or automatically.

The second automated dispenser 318 may selectively dispense the second amount of adhesive 320 onto the sponge 308 at the defined location 316. The location and/or orientation of the sponge 308 at the third location 332 of the assembly surface 310 may be determined using, for example, a sensor 330 that may be oriented to detect objects at the third location 332. The second automated dispenser 318 may be physically coupled to a second appendage 319 that may be selectively movable with multiple degrees of freedom. The second automated dispenser 318 may thereby be positioned using the second appendage 319 relative to the sponge 308 located at the third location 332 to deposit the second amount of adhesive 320. The second amount of adhesive 320 deposited by the second automated dispenser 318 may be used to cover, at least partially and preferably completely, the wireless transponder 314 at the defined location 316. As such, a combination of the first amount of adhesive 306 and the second amount of adhesive 320 may thereby enclose the wireless transponder 314. In some implementations, the second amount of adhesive 320 may be made onto the first amount of adhesive 306, to partially or completely cover the first amount of adhesive 306.

In some implementations, the second amount of adhesive 320 may be the same type of adhesive as the first amount of adhesive 306. Alternatively, in some implementations, the second amount of adhesive 320 may be a different type of adhesive than the first amount of adhesive 306. For example, the first amount of adhesive 306 may be an adhesive that has desirable adhesive qualities with regard to the material that comprises the sponge 308, and the second amount of adhesive 320 may be a type of adhesive that provides desirable protective qualities (e.g., hardness) to protect the wireless transponder 314.

The second amount of adhesive 320 may include one or more types of adhesive that are approved for use in a surgical environment. In some implementations, for example, the second amount of adhesive 320 may be biocompatible, permitting use in vivo. The second amount of adhesive 320 may, for example, take the form of an adhesive web film. The second amount of adhesive 320 may, for example, take the form of a thermal lamination film. The second amount of adhesive 320 may, for example, take the form of a meltable plastic layer, such as, for example, a thermoplastic layer. The second amount of adhesive 320 may be a thermosetting plastic that has an initial cure temperature at which the thermosetting plastic layer cures, such as, for example, a temperature less than 130 degrees Centigrade. The second amount of adhesive 320 may be a heat-activated adhesive layer. Alternatively or additionally, the second amount of adhesive 320 may be pressure-activated adhesive layer or a pressure-sensitive adhesive layer. Alternatively or additionally, the second amount of adhesive 320 may be a water-activated adhesive. Alternatively or additionally, the second amount of adhesive 320 may be a radiation curing adhesive that may be cured via illuminating the second deposit of adhesive with one or more types of radiation (e.g., ultraviolet (“UV”) light, and visible light) for a period of time. The second amount of adhesive 320 may include, for example, acrylate resins (e.g., acrylate epoxies, polyurethanes, and silicones), photoinitiators, photosensitizers, and/or monomers.

After the second amount of adhesive 320 is deposited onto the sponge 308 at the fourth location 344, the assembly surface 310 may advance the sponge 308 towards the illumination source 322 located at a fifth location 346. The illumination source 322 may emit a type of radiation that may be used to cure one or both of the first amount of adhesive 306 and the second amount of adhesive 320. The illumination source 322, for example, may emit radiation that is within the UV spectrum and/or within the visible light spectrum. The radiation from the illumination source 322 may be incident on the defined portion 316 of the sponge 308 for a defined period of time that is sufficient to cure either or both of the first amount of adhesive 306 and the second amount of adhesive 320. Alternatively, or in addition, the illumination source 322 may include a heat source that heats the defined location 316 of the sponge 308 to a specified temperature for a period of time that may be sufficient to cure one or both of the first amount of adhesive 306 and the second amount of adhesive 320. After one or both of the first amount of adhesive 306 and the second amount of adhesive 320 are cured, the assembly surface 310 conveys the sponge 308 for further processing and/or packaging.

FIG. 3B shows an assembly system 350 that includes a processing surface 352 that may be used to produce wireless detectable sponges, in which the processing station 352 includes one or more automated dispensers 354 that dispense adhesive onto a sponge 358 at a defined location 360, and at least one robotic appendage 362 that picks and places wireless transponders 364 onto the sponges 358 at a defined location 360, according to at least one illustrated implementation. The sponge 358 may be placed onto the processing surface 352 manually or by using an automated component or tool, such as, for example, an automated peel that may be used to place the sponge 358 onto, and retrieve the sponge 358 from, the processing surface 352. In some implementations, the assembly system 350 may include one or more sensors 365 that may be used to detect a position and/or orientation of the sponges 358 on the processing surface 352. Such sensors 365 may include, for example, image sensors (e.g., imagers, cameras) that capture one or more images of portions of the processing surface 352 and/or emitter-receiver pairs that may be used to detect the presence or absence of sponges 358 at one or more positions, as well as the orientation of detected sponges 358, along the processing surface 352.

The automated dispenser 354 may be physically coupled to an appendage 366 that may be selectively movable with multiple degrees of freedom. In some implementations, the automated dispenser 354 may include one or more dispensing nozzles to dispense adhesive. In some implementations, for example, a first dispensing nozzle 368 on the automated dispenser 354 may dispense a first type of adhesive, and a second dispensing nozzle 370 on the automated dispenser 354 may dispense a second type of adhesive. When the sponge 358 is detected on the processing surface 352 (via, for example, one or more sensors 365), the automated dispenser 354 may be positioned using the appendage 366 relative to the sponge 358 to dispense a first amount of adhesive 372 onto the sponge 358 at the defined location 360.

The first amount of adhesive 372 may include one or more types of adhesive that are approved for use in a surgical environment. In some implementations, for example, the first amount of adhesive 372 may be biocompatible, permitting use in vivo. The first amount of adhesive 372 may, for example, take the form of an adhesive web film. The first amount of adhesive 372 may, for example, take the form of a thermal lamination film. The first amount of adhesive 372 may, for example, take the form of a meltable plastic layer, such as, for example, a thermoplastic layer. The first amount of adhesive 372 may be a thermosetting plastic that has an initial cure temperature at which the thermosetting plastic layer cures, such as, for example, a temperature less than 130 degrees Centigrade. The first amount of adhesive 372 may be a heat-activated adhesive layer. Alternatively or additionally, the first amount of adhesive 372 may be pressure-activated adhesive layer or a pressure-sensitive adhesive layer. Alternatively or additionally, the first amount of adhesive 372 may be a water-activated adhesive. Alternatively or additionally, the first amount of adhesive 372 may be a radiation curing adhesive that may be cured via illuminating the second deposit of adhesive with one or more types of radiation (e.g., ultraviolet (“UV”) light, and visible light) for a period of time. The first amount of adhesive 372 may include, for example, acrylate resins (e.g., acrylate epoxies, polyurethanes, and silicones), photoinitiators, photosensitizers, and/or monomers.

Once the first amount of adhesive 372 is deposited, the robotic appendage 362 may be used to pick wireless transponders 364 from a source 374 of wireless transponders, and place individual wireless transponders 364 onto the sponge 358 at the defined location 360. The source 374 of wireless transponders may include a storage container 376 in which the wireless transponders 364 may be stored vertically in defined slots within the storage container 376. The robotic appendage 362 may use an end-of-arm tool to pick one of the wireless transponders 364 at a first location that corresponds to a one of the defined slots in the storage container 376. Once the robotic appendage 362 picks the wireless transponder 364, the robotic appendage 362 may move towards a second position that may correspond or be related to the defined location 360 of the sponge 358. The robotic appendage 362 may then place the wireless transponder 364 onto the first amount of adhesive 356 of the sponge 358. The robotic appendage 362 may then move back towards the storage container 376 to prepare to pick the next wireless transponder 364. In such an implementation, the robotic appendage 362 may be used to place one or multiple wireless transponders 364 onto the sponge 358.

Once the robotic appendage 362 has completed placing one or more wireless transponders 364 on the sponge 358, the automated dispenser 354 may be positioned using the appendage 366 relative to the sponge 358 to dispense a second amount of adhesive (not shown) onto the sponge 358 at the defined location 360. In some implementations, the second amount of adhesive may be the same type of adhesive as the first amount of adhesive. Alternatively, the second amount of adhesive may be a different type of adhesive from the first amount of adhesive 356.

The second amount of adhesive may include one or more types of adhesive that are approved for use in a surgical environment. In some implementations, for example, the second amount of adhesive may be biocompatible, permitting use in vivo. The second amount of adhesive may, for example, take the form of an adhesive web film. The second amount of adhesive may, for example, take the form of a thermal lamination film. The second amount of adhesive may, for example, take the form of a meltable plastic layer, such as, for example, a thermoplastic layer. The second amount of adhesive may be a thermosetting plastic that has an initial cure temperature at which the thermosetting plastic layer cures, such as, for example, a temperature less than 130 degrees Centigrade. The second amount of adhesive may be a heat-activated adhesive layer. Alternatively or additionally, the second amount of adhesive may be pressure-activated adhesive layer or a pressure-sensitive adhesive layer. Alternatively or additionally, the second amount of adhesive may be a water-activated adhesive. Alternatively or additionally, the second amount of adhesive may be a radiation curing adhesive that may be cured via illuminating the second deposit of adhesive with one or more types of radiation (e.g., ultraviolet (“UV”) light, and visible light) for a period of time. The second amount of adhesive may include, for example, acrylate resins (e.g., acrylate epoxies, polyurethanes, and silicones), photoinitiators, photosensitizers, and/or monomers.

The assembly system 350 may include an illumination source 378 that may be used to illuminate a portion of the sponge 358, such as, for example, the defined location 360, with radiation. The radiation from the illumination source 378 may be used to cure one or both of the first amount of adhesive 306 and the second amount of adhesive 320. In some implementations, the illumination source may emit radiation after the first amount of adhesive 356 is deposited onto the sponge 358 but before the second amount of adhesive is deposited, to thereby cure the first amount of adhesive 356 during a first time period. In such an implementation, the illumination source 378 may be turned OFF, for example, when the wireless transponder 364 is being picked and placed, and then turned ON after the second amount of adhesive is deposited onto the defined location 360. The radiation from the illumination source 378 may thereby cure the second amount of adhesive during a second time period that occurs after the first time period.

In some implementations, the illumination source 378 may emit radiation that is within the UV spectrum and/or within the visible light spectrum. The radiation from the illumination source 378 may be incident on the defined location 360 of the sponge 358 for a defined period of time that is sufficient to cure either or both of the first amount of adhesive 356 and the second amount of adhesive. Alternatively, or in addition, the illumination source 322 may include a heat source that heats the defined location 360 of the sponge 358 to a specified temperature for a period of time that may be sufficient to cure one or both of the first amount of adhesive 356 and the second amount of adhesive. After one or both of the first amount of adhesive 356 and the second amount of adhesive are cured, the sponge 358 may be removed from the processing surface 352 for further processing and/or packaging.

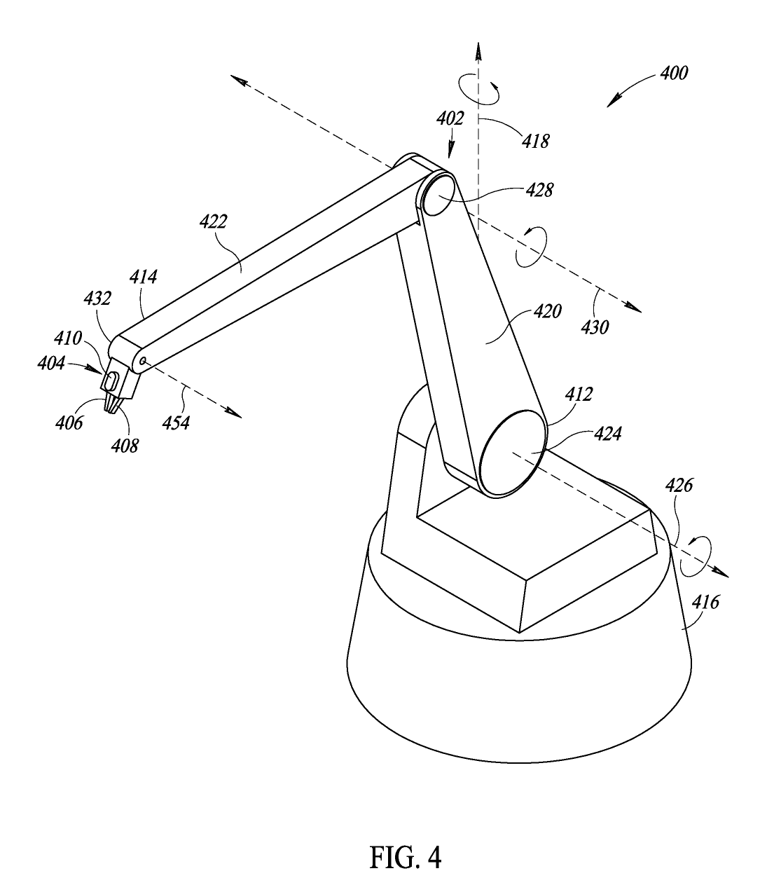

FIG. 4 shows a dispenser 400 that may be used to dispense adhesive, according to at least one illustrated implementation. The dispenser 400 may include an appendage 402 and an end-of-arm tool 404 in which the end-of-arm tool 404 includes a first nozzle 406 and a second nozzle 408. The first nozzle 406 and the second nozzle 408 may be used to deposit the same type of adhesive and/or to deposit different types of adhesive. In some implementations, the end-of-arm tool 404 may include an imager 410 that has a field of view that extends outward from the imager 410 towards the first nozzle 406 and/or the second nozzle 408. The imager 410 may capture one or more images that may be used to determine the presence or absence of a sponge, and to determine if the first nozzle 406 and/or the second nozzle 408 are appropriately positioned to deposit adhesive.

The appendage 402 may extend from a proximal end 412 to a distal end 414. The proximal end 412 of the appendage 402 may be physically coupled to a rotatable platform 416 that provides a vertical axis of rotation 418 for the appendage 402. The vertical axis of rotation 418 may thereby be used to position the distal end 414 of the appendage 402 with respect to a sponge to be processed. The rotatable platform 416 may be drivingly coupled to a motor (not shown). In some implementations, the motor may rotate the rotatable platform 416 about 360°, a plurality of times, without restriction. In some implementations, the rotation of the rotatable platform 416 may be restricted such that the rotatable platform 416 may rotate less than 360° (e.g., 180°, 90°, 45°). Such restrictions on rotation may be used, for example, to protect electrical, fluidic, or other connections that extend to the appendage 402 from being damaged.

The appendage 402 may include a plurality of robotic arms, such as, for example a first robotic arm 420 and a second robotic arm 422. The first robotic arm 420 may be located relatively towards the proximal end 412 of the appendage 402, and the second robotic arm 422 may be located relatively towards the distal end 414 of the appendage 402. The first robotic arm 420 may rotatably couple with the rotatable platform 416 at a first joint 424 that provides a first robotic arm axis of rotation 426 that extends horizontally outward from the first joint 424. In some implementations, the rotation of the first robotic arm 420 of the appendage 402 about the first robotic arm axis of rotation 426 may be controlled, for example, by one or more types of motors, such as a stepper motor, that may be used to control the location and/or the rate of rotation of the first robotic arm 420 about the first robotic arm axis of rotation 426.

The second robotic arm 422 may be rotatably coupled to the first robotic arm 420 by a second joint 428 that provides a second robotic arm axis of rotation 430 that extends laterally outward in a direction that is perpendicular to each of the first robotic arm 420 and the second robotic arm 422. The rotation of the second robotic arm 422 of the appendage 402 about the second robotic arm axis of rotation 430 may be controlled, for example, by one or more types of motors, such as a stepper motor, that may be used to control the location and/or the rate of rotation of the second robotic arm 422 about the second robotic arm axis of rotation 430. The end-of-arm tool 404 may be rotatably coupled to the second robotic arm 422 via a third joint 432 that provides a third axis of rotation 434 that extends outward in a direction that is perpendicular to a length of the second robotic arm 422. Such a third joint 432 enables the end-of-arm tool 404 to rotate with respect to one end of the second robotic arm 422. The rotation of the end-of-arm tool 404 of the appendage 402 may be controlled, for example, by one or more types of motors, such as a stepper motor, that may be used to control the location and/or the rate of rotation of the end-of-arm tool 404 about the third axis of rotation 434.

FIG. 5A shows a pick-and-place component 500 that includes a robotic appendage 502 that picks and places wireless transponders 504 from a supply of wireless transponders 506 that are delivered via a tape-and-reel 508, according to at least one illustrated implementation. The tape-and-reel 508 may be comprised of a reel (not shown) around which a length of tape 510 is wound. The tape 510 may be unwound from the reel in a direction towards the robotic appendage 502. As such, the wireless transponder 506 to be picked by the robotic appendage 502 may be located at a dispensing location 512 that is proximate the robotic appendage 502. A plurality of wireless transponders 506 may be attached to the length of tape 510, in which each wireless transponder 506 is located at a defined distance 514 from each adjacent wireless transponder 506. In such an implementation, the length of tape 510 may be advanced by such a defined distance 514 once each wireless transponder 506 is removed from the length of tape 510 by the robotic appendage 502. When the length of tape 510 advances, the now-empty portion of the tape may be directed towards a return slot 516 that may be used to collect the empty tape (e.g., using a collection reel around which the used length of tape 510 is wound).

The robotic appendage 502 may include an end-of-arm tool 518 that may be used to pick the wireless transponders 504 from the length of tape 510. In some implementations, such as shown in FIG. 5A, the end-of-arm tool 518 may include two or more opposable mechanical digits 520 that are selectively movable towards and away from each other. Such opposable mechanical digits 520 may be selectively moved towards each other in order to retrieve or pick and move with a wireless transponder 506. The opposable mechanical digits 520 may be selectively moved away from each other to place the wireless transponders 506 to drop or deposit the wireless transponders 506. In some implementations, as discussed below, the end-of-arm tool 518 may include a resiliently conformable member that has an associated port. The port may be fluidly coupled to a source of low pressure (e.g., a vacuum) that may be used to create suction at the port such that the end-of-arm tool 518 may retrieve or pick each wireless transponder 504. The low pressure source may be disengaged to remove the vacuum at the end-of-arm tool 518 thereby releasing the wireless transponder 504 so that the wireless transponder 504 may be placed.

The wireless transponder 504 may be comprised of one or more RFID transponders or RFID tags that encode unique identifiers that may uniquely identify the wireless transponder 504. The wireless transponder 504 may be comprised of one or more RFID transponders or RFID tags that encode unique identifiers that may uniquely identify the wireless transponder 504. Alternatively, or in addition, the wireless transponder 504 may be comprised of one or more dumb transponders that do not encode unique identifiers, but may return a signal that may be used determine at least one of a presence or absence of the wireless communications dumb transponders in the range of a wireless communications presence/absence interrogation system(s).

The robotic appendage 502 may extend from a proximal end 522 to a distal end 524. The proximal end 522 of the robotic appendage 502 may be physically coupled to a rotatable platform 526 that provides a vertical axis of rotation 527 for the robotic appendage 502. The vertical axis of rotation 527 may thereby be used to position the distal end 524 of the robotic appendage 502 with respect to a sponge to be processed. The rotatable platform 526 may be drivingly coupled to a motor (not shown). In some implementations, the motor may rotate the rotatable platform 526 about 360°, a plurality of times, without restriction. In some implementations, the rotation of the rotatable platform 526 may be restricted such that the rotatable platform 526 may rotate less than 360° (e.g., 180°, 90°, 45°).

The robotic appendage 502 may include a plurality of segments, also referred to as robotic arms, such as, for example a first robotic arm 528 and a second robotic arm 530. The first robotic arm 528 may be located relatively towards the proximal end 522 of the robotic appendage 502, and the second robotic arm 530 may be located relatively towards the distal end 524 of the robotic appendage 502. The first robotic arm 528 may rotatably couple with the rotatable platform 526 at a first joint 532 that provides a first robotic arm axis of rotation 534 that extends horizontally outward from the first joint 532. In some implementations, the rotation of the first robotic arm 528 of the robotic appendage 502 about the first robotic arm axis of rotation 534 may be controlled, for example, by one or more types of motors, such as a stepper motor, that may be used to control the location and/or the rate of rotation of the first robotic arm 528 about the first robotic arm axis of rotation 534.

The second robotic arm 530 may be rotatably coupled to the first robotic arm 528 by a second joint 536 that provides a second robotic arm axis of rotation 538 that extends laterally outward in a direction that is perpendicular to each of the first robotic arm 528 and the second robotic arm 530. The rotation of the second robotic arm 530 of the robotic appendage 502 about the second robotic arm axis of rotation 538 may be controlled, for example, by one or more types of motors, such as a stepper motor, that may be used to control the location and/or the rate of rotation of the second robotic arm 530 about the second robotic arm axis of rotation 538. The end-of-arm tool 518 may be rotatably coupled to the second robotic arm 530 via a third joint 540 that provides a third axis of rotation 542 that extends outward in a direction that is perpendicular to a length of the second robotic arm 530. Such a third joint 540 enables the end-of-arm tool 518 to rotate with respect to one end of the second robotic arm 530. The rotation of the end-of-arm tool 518 of the robotic appendage 502 may be controlled, for example, by one or more types of motors, such as a stepper motor, that may be used to control the location and/or the rate of rotation of the end-of-arm tool 518 about the third axis of rotation 542.

FIG. 5B shows a pick-and-place component 550 that include the robotic appendage 502 that picks and places wireless transponders 504 from a supply 552 of wireless transponders contained within a storage container 554, according to at least one illustrated implementation. The storage container 554 may include a plurality of slots 556 that may each hold a wireless transponder 504. In some implementations, the slots 556 may be used to hold the wireless transponders 504 in a vertical direction to facilitate the picking of each wireless transponder 504 by the end-of-arm tool 518 on the robotic appendage 502. As shown in FIG. 5B, the end-of-arm tool 518 may include a conformable member 558 that may have an associated port 560. The conformable member 558 may be fluidly coupled to a source of a pressure (e.g., a vacuum) that may be used to create suction at the associated port 560, as discussed above. The end-of-arm tool 518 may be used to retrieve and transport items when suction is present at the associated port 560. The slots 556 may be arranged within a plurality of rows and columns within the storage container 554, such that the slots 556 may be arranged at regular, defined distances within each row and column. The regular, defined arrangement of the slots 556 may be used to facilitate the retrieval or picking of the wireless transponders 504 from the storage container 554 by the robotic appendage 502.

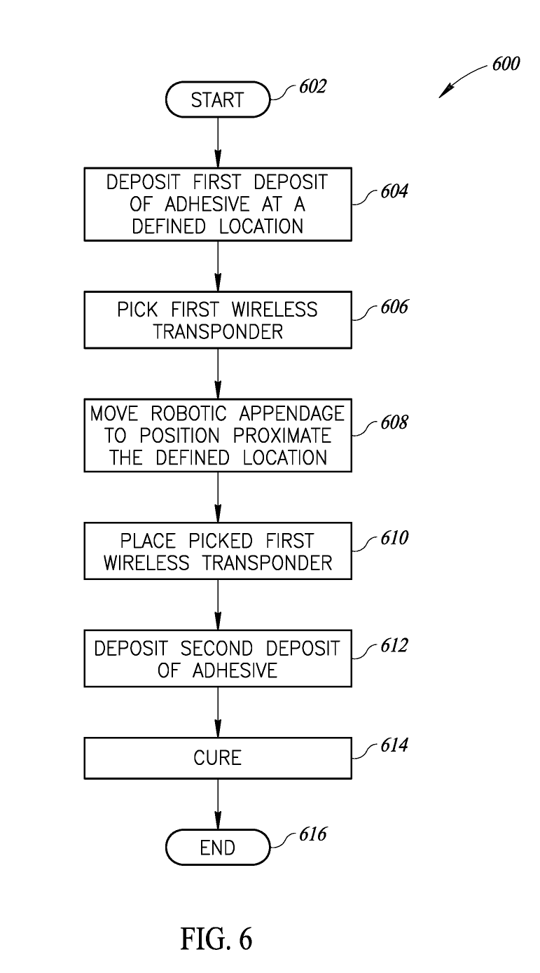

FIG. 6 shows a method 600 for manufacturing wireless detectable sponges, according to at least one illustrated implementation. The method 600 starts at 602, for example, on powering up the assembly system 300 and/or the assembly system 350.

At 604, an automated dispenser deposits a first deposit of adhesive onto a sponge at a defined location. In some implementations, the automated dispenser may be carried by a robotic appendage that may be used to position the automated dispenser relative to the defined location. The first amount of adhesive may include one or more types of adhesive that are approved for use in a surgical environment. In some implementations, for example, the first amount of adhesive may be biocompatible, permitting use in vivo. The first amount of adhesive may, for example, take the form of an adhesive web film. The first amount of adhesive may, for example, take the form of a thermal lamination film. The first amount of adhesive may, for example, take the form of a meltable plastic layer, such as, for example, a thermoplastic layer. The first amount of adhesive may be a thermosetting plastic that has an initial cure temperature at which the thermosetting plastic layer cures, such as, for example, a temperature less than 130 degrees Centigrade. The first amount of adhesive may be a heat-activated adhesive layer. Alternatively or additionally, the first amount of adhesive may be pressure-activated adhesive layer or a pressure-sensitive adhesive layer. Alternatively or additionally, the first amount of adhesive may be a water-activated adhesive. Alternatively or additionally, the first amount of adhesive may be a radiation curing adhesive that may be cured via illuminating the second deposit of adhesive with one or more types of radiation (e.g., ultraviolet (“UV”) light, and visible light) for a period of time. The first amount of adhesive may include, for example, acrylate resins (e.g., acrylate epoxies, polyurethanes, and silicones), photoinitiators, photosensitizers, and/or monomers.

At 606, a robotic appendage picks a wireless transponder from a supply of wireless transponders. The wireless transponder may be picked from a first position proximate the source of wireless transponders. In some implementations, picking the wireless transponder may include moving the robotic arm to position the end-of-arm tool with respect to a source of wireless transponders. Such a source of wireless transponders may include, for example, a tape-and-reel that holds a plurality of wireless transponders along a length of tape, and/or a storage container that may be used to hold a plurality of wireless transponders. The wireless transponder may be comprised of one or more RFID transponders or RFID tags that encode unique identifiers that may uniquely identify the wireless transponder. Alternatively, or in addition, the wireless transponder may be comprised of one or more dumb transponders that do not encode unique identifiers, but may return a signal that may be used determine at least one of a presence or absence of the wireless communications dumb transponders in the range of a wireless communications presence/absence interrogation system(s).

At 608, the robotic appendage is moved from the first position to a second position proximate the defined location. In some implementations, one or more imagers and/or sensors may be used to facilitate the movement of the robotic appendage.

At 610, the robotic appendage places the picked wireless transponder onto the first deposit of adhesive located at the defined location on the sponge. In some implementations, the wireless transponder may make a depression or groove within the first deposit of adhesive when the wireless transponder is placed onto the first deposit of adhesive. In some implementations, the sponge may have a length, a width, and a thickness, such that the picked wireless transponder may be placed at the defined location in a defined orientation relative to the length and/or the width of the sponge. For example, in some implementations in which the wireless transponder is located proximate an edge of the sponge, the wireless transponder may be oriented to extend parallel to the edge of the sponge. In some implementations in which the wireless transponder is located proximate a corner formed by two intersecting edges of the sponge, the wireless transponder may be oriented to extend perpendicular to a line that bisects the angle formed by the two intersecting edges of the sponge.

At 612, an automated dispenser deposits a second deposit of adhesive onto the sponge at the defined location. In some implementations, the automated dispenser may be carried by a robotic appendage that may be used to position the automated dispenser relative to the defined location. In some implementations, the second deposit of adhesive may cover the wireless transponder that has been placed onto the sponge at the defined location. In such an implementation, the first deposit of adhesive and the second deposit of adhesive may combine to enclose the wireless transponder. In some implementations, the automated dispenser that deposits the second deposit of adhesive may the same automated dispenser that deposits the first deposit of adhesive. Alternatively, the automated dispenser that deposits the second deposit of adhesive may be different from the automated dispenser that deposits the first deposit of adhesive. In some implementations, the first deposit of adhesive may be the same type of adhesive as the second deposit of adhesive. Alternatively, the first deposit of adhesive may be a type of adhesive that is different from the second type of adhesive.

At 614, at least one of the first deposit of adhesive and/or the second deposit of adhesive are cured. In some implementations, an illumination source may be used to illuminate at least a portion of the defined location with radiation within a defined range for a defined period of time to thereby cure at least one of the first deposit of adhesive and/or the second deposit of adhesive. The defined range for the radiation may include, for example, radiation in the UV spectrum and/or radiation in the visible light spectrum. Curing the first deposit of adhesive and/or the second deposit of adhesive may be used to encapsulate the wireless transponder and to attach the wireless transponder to the sponge. Alternatively, or in addition, the illumination source may include a heat source that heats the defined location of the sponge to a specified temperature for a period of time that may be sufficient to cure one or both of the first amount of adhesive and the second amount of adhesive.

At 616, the method 600 terminates until invoked again.

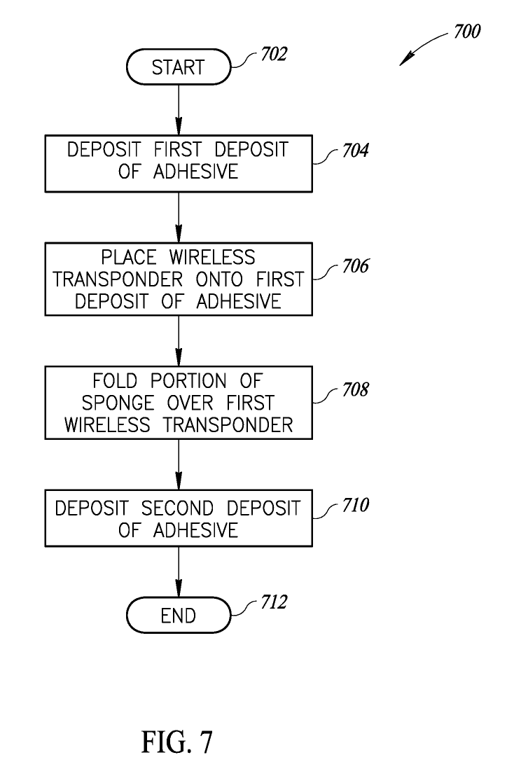

FIG. 7 shows a method 700 for folding a portion of the sponge as part of manufacturing a wireless detectable sponge, according to at least one illustrated implementation. The method 700 starts at 702, for example, on powering up the assembly system 300 and/or the assembly system 350.

At 704, a robotic appendage may place the wireless transponder onto the first deposit of adhesive at the defined location. In some implementations, the sponge may have a length, a width, and a thickness, such that the picked wireless transponder may be placed at the defined location in a defined orientation relative to the length and/or the width of the sponge. For example, in some implementations in which the wireless transponder is located proximate an edge of the sponge, the wireless transponder may be oriented to extend parallel to the edge of the sponge. In some implementations in which the wireless transponder is located proximate a corner formed by two intersecting edges of the sponge, the wireless transponder may be oriented to extend perpendicular to a line that bisects the angle formed by the two intersecting edges of the sponge.

At 706, at least a portion of the sponge may be folded to cover the wireless transponder placed at the defined location. In such an implementation, the amount of sponge that is folded may be minimized such that the folded portion of the sponge just covers the defined location. In some implementations, the folding may be performed manually. In some implementations, the folding may be performed by an automated machine.

At 708, an automated dispenser deposits a second deposit of adhesive over the folded portion of the sponge. In such an implementation, the wireless transponder may be enclosed by a combination of the first deposit of adhesive, the second deposit of adhesive, and the portion of the sponge folded over the defined location.

At 710, the method 700 terminates until invoked again. The method 700 may be performed, for example, as part of the method 600.

Transponders useful for marking medical procedure related objects may take a variety of forms. Transponders capable of withstanding sterilization procedures would be particularly advantageous. A permanent memory type RFID transponder which retains information or data, for instance a unique identifier, and which is substantially gamma ray resistant and capable of being subjected to the relatively high temperatures often associated with sterilization may be formed from an antenna, passive power or backscatter circuit and a permanent memory circuit communicatively coupled to the antenna and powered via the passive power or backscatter circuit to transmit the contents of the permanent memory in response to power derived from an interrogation signal. The permanent memory circuit may advantageously take the form or may incorporate aspects of the permanent memory circuits described in one or more of U.S. Pat. Nos. 7,609,538; 7,471,541; 7,269,047; 7,042,722; 7,031,209; 6,992,925; 6,972,986; 6,956,258; 6,940,751; 6,898,116; 6,856,540; 6,822,888; 6,798,693; 6,791,891; 6,777,757; 6,766,960; 6,700,151; 6,671,040; 6,667,902; and 6,650,143, all of which are incorporated herein by reference in their entireties to the extent that such are not inconsistent with the other portions of present detailed description. Applicants have recognized that such permanent memory circuits may be resistant to gamma ray radiation, chemicals (e.g., peroxide) and/or high temperatures, and thus may be particularly suitable for use in manufacturing transponders for use in marking objects that will be subjected to the extremes of sterilization. The permanent memory type transponder may include a housing, shell or encapsulant. Such a permanent memory transponder may be particularly useful for marking gauze or sponges. Such a transponder may be attached to a medical procedure related object in any variety of fashions, including sewn to, sewn in, adhered via adhesives or heat or RF welding, riveted, tied to, via a snap, stapled, etc.

The above description of illustrated embodiments, including what is described in the Abstract, is not intended to be exhaustive or to limit the embodiments to the precise forms disclosed. Although specific embodiments of and examples are described herein for illustrative purposes, various equivalent modifications can be made without departing from the spirit and scope of the disclosure, as will be recognized by those skilled in the relevant art. The teachings provided herein of the various embodiments can be applied to other transponders and interrogation and detection systems, not necessarily the exemplary surgical object transponders and interrogation and detection systems generally described above.

Also for instance, many of the embodiments described herein, perform interrogation and detection of transponder tagged objects using multiple antennas. Successive ones of the antennas may be used to transmit an interrogation signal, while two or more antennas are monitored for a response to the interrogation signal. Such may provide significant advantages over more conventional methods, for example motion-based methods that employ motion (e.g., sweeping) of an antenna (e.g., wand) over a patient. For instance, this allows the transmit and receive paths to the transponder to be different from one another (e.g., the transmit path is from a first antenna to a transponder, while the receive path is from the transponder to a second antenna). Hence, the path length to the transponder may be shortened in many configurations, thus improving the signal. For instance, when using a single antenna to both transmit an interrogation signal and to receive a response to the interrogation signal, the power of the received signal is equal to about the 6th root of the input power. However, when using multiple antennas to transmit and receive over the same area, interrogation path length in one direction may be shorter. Another advantage is that all scan time may be averaged, allowing a longer noise time averaging (e.g., 10 seconds) as opposed to motion-based scanning, where integration time may be limited (e.g., about 0.25 seconds per sample). Even further, a representative value of noise samples measured over a plurality of antennas may be employed to determine noise to be removed from noise plus signals received at one of the antennas, thereby advantageously lowering a noise floor and/or increasing range or performance. Thus, the various disclosed embodiments may provide significantly better performance.

While generally discussed in terms of a passive wireless transponder, which requires an interrogation signal to derive electrical energy to power operation, for example to backscatter a response signal, such is not necessary to all implementations. For example, some implementations can employ an active transponder, with an onboard, consumable power source (e.g., chemical battery), which can emit signals from time-to-time (e.g., periodically) without any external stimulus (e.g., interrogation signals). Such implementations are of course subject to the power source being capable of operating over long times, even if the object to which the active wireless transponder is attached is not put into service for several years. Thus, most implementations will employ passive wireless transponders, and thus employ interrogation signals.

Also for instance, the foregoing detailed description has set forth various embodiments of the devices and/or processes via the use of block diagrams, schematics, and examples. Insofar as such block diagrams, schematics, and examples contain one or more functions and/or operations, it will be understood by those skilled in the art that each function and/or operation within such block diagrams, flowcharts, or examples can be implemented, individually and/or collectively, by a wide range of hardware, software, firmware, or virtually any combination thereof. In one embodiment, the present subject matter may be implemented via Application Specific Integrated Circuits (ASICs). However, those skilled in the art will recognize that the embodiments disclosed herein, in whole or in part, can be equivalently implemented in standard integrated circuits, as one or more computer programs running on one or more computers (e.g., as one or more programs running on one or more computer systems), as one or more programs running on one or more controllers (e.g., microcontrollers) as one or more programs running on one or more processors (e.g., microprocessors), as firmware, or as virtually any combination thereof, and that designing the circuitry and/or writing the code for the software and or firmware would be well within the skill of one of ordinary skill in the art in light of this disclosure.

Various exemplary methods or processes are described. It is noted that these exemplary methods or processes may include additional acts and/or may omit some acts. In some implementations, the acts of the various exemplary methods or processes may be performed in a different order and/or some acts may be executed or performed concurrently.

In addition, those skilled in the art will appreciate that the mechanisms as taught herein are capable of being distributed as a program product in a variety of forms, and that an illustrative embodiment applies equally regardless of the particular type of physical signal bearing media used to actually carry out the distribution. Examples of signal bearing media include, but are not limited to, the following: recordable type media such as floppy disks, hard disk drives, CD ROMs, digital tape, and computer memory.

The various embodiments described above can be combined to provide further embodiments. To the extent not inconsistent with the teachings herein, all U.S. patents, U.S. patent application publications, U.S. patent applications, foreign patents, foreign patent applications and non-patent publications commonly owned with this patent application and referred to in this specification and/or listed in the Application Data Sheet including: U.S. Pat. No. 6,026,818, issued Feb. 22, 2000; U.S. Patent Publication No. US 2004/0250819, published Dec. 16, 2004; U.S. Pat. No. 8,710,957, issued Apr. 29, 2014; U.S. Pat. No. 7,898,420, issued Mar. 1, 2011; U.S. Pat. No. 7,696,877, issued Apr. 13, 2010; U.S. Pat. No. 8,358,212, issued Jan. 22, 2013; U.S. Pat. No. 8,111,162, issued Feb. 7, 2012; U.S. Pat. No. 8,354,931, issued Jan. 15, 2013; U.S. Patent Publication No. US 2010/0108079, published May 6, 2010; U.S. Patent Publication No. US 2010/0109848, published May 6, 2010; U.S. Patent Publication No. US 2011/0004276, published Jan. 6, 2011; U.S. Patent Publication No. US 2011/0181394, published Jul. 28, 2011; U.S. Patent Publication No. US 2013/0016021, published Jan. 17, 2013; PCT Patent Publication No. WO 2015/152975, published Oct. 8, 2015; U.S. Provisional patent application Ser. No. 62/143,726 filed Apr. 6, 2015; U.S. Provisional patent application Ser. No. 62/182,294 filed Jun. 19, 2015; U.S. Provisional patent application Ser. No. 62/164,412 filed May 20, 2015; U.S. Non-Provisional patent application Ser. No. 14/523,089 filed Oct. 24, 2014; U.S. Non-Provisional patent application Ser. No. 14/327,208 filed Jul. 9, 2014; U.S. Non-Provisional patent application Ser. No. 15/003,515 filed Jan. 21, 2016; U.S. Non-Provisional patent application Ser. No. 15/003,524 filed Jan. 21, 2016; U.S. Non-Provisional patent application Ser. No. 15/052,125 filed Feb. 24, 2016; U.S. Non-Provisional patent application Ser. No. 15/053,965 filed Feb. 25, 2016; U.S. Provisional patent application Ser. No. 62/360,864 filed Jul. 11, 2016 and entitled “METHOD AND APPARATUS TO ACCOUNT FOR TRANSPONDER TAGGED OBJECTS USED DURING CLINICAL PROCEDURES, EMPLOYING A SHIELDED RECEPTACLE”; U.S. Provisional patent application Ser. No. 62/360,866 filed Jul. 11, 2016 and entitled “METHOD AND APPARATUS TO ACCOUNT FOR TRANSPONDER TAGGED OBJECTS USED DURING CLINICAL PROCEDURES EMPLOYING A SHIELDED RECEPTACLE WITH ANTENNA”; and U.S. Provisional patent application Ser. No. 62/360,868 filed Jul. 11, 2016 and entitled “METHOD AND APPARATUS TO ACCOUNT FOR TRANSPONDER TAGGED OBJECTS USED DURING CLINICAL PROCEDURES, FOR EXAMPLE INCLUDING COUNT IN AND/OR COUNT OUT AND PRESENCE DETECTION”, are each incorporated herein by reference, in their entirety. Aspects of the embodiments can be modified, if necessary, to employ systems, circuits and concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A process to manufacture wirelessly detectable sponges for use in medical procedures, the process comprising:
depositing a first deposit of adhesive onto a first sponge at a first defined location on the first sponge;
picking a first wireless transponder by a robotic appendage from a supply of wireless transponders;
moving the robotic appendage to position the picked first wireless transponder at least proximate the first defined location on the first sponge;
placing the picked first wireless transponder onto the first deposit of adhesive at the first defined location on the first sponge;
depositing a second deposit of adhesive to cover the wireless transponder; and
curing at least one of the first or the second deposits of adhesive.

2. The process of claim 1, wherein the first sponge has a length, a width and a thickness, and placing the picked first wireless transponder onto the first deposit of adhesive at the first defined location on the first sponge includes placing the picked first wireless transponder onto the first deposit of adhesive at the first defined location on the first sponge in a defined orientation relative to the length and width of the first sponge.

3. The process of claim 1, wherein depositing a second deposit of adhesive to cover the wireless transponder includes depositing the second deposit of adhesive to enclose the wireless transponder by a combination of the first and the second deposits of adhesive.

4. The process of claim 1, wherein the robotic appendage is a robotic arm that includes an end-of-arm tool, and picking a first wireless transponder by a robotic appendage includes moving the robotic arm to position the end-of-arm tool with respect to a storage container that holds the supply of wireless transponders.

5. The process of claim 1, wherein depositing a first deposit of adhesive onto a first sponge at a first defined location on the first sponge includes depositing the first deposit of adhesive onto the first sponge at the first defined location on the first sponge via a first automated dispenser.

6. The process of claim 5, wherein the automated dispenser is carried by a robotic appendage and depositing the first deposit of adhesive onto the first sponge at the first defined location on the first sponge via an automated dispenser includes positioning the automated dispenser relative to the first sponge by the robotic appendage.

7. The process of claim 5, wherein depositing a second deposit of adhesive to cover the wireless transponder includes depositing the second deposit of adhesive via the first automated dispenser.

8. The process of claim 5, wherein depositing a second deposit of adhesive to cover the wireless transponder includes depositing the second deposit of adhesive via a second automated dispenser.

9. The process of claim 5, wherein depositing a first deposit of adhesive onto a first sponge at a first defined location on the first sponge includes depositing a first amount of a first type of adhesive onto the first sponge at the first defined location on the first sponge, and wherein depositing a second deposit of adhesive to cover the wireless transponder includes depositing a second amount of the first type of adhesive to cover the wireless transponder.

10. The process of claim 1, further comprising:
folding a portion of the first sponge to cover the first transponder before depositing the second deposit of adhesive to cover the wireless transponder.

11. The process of claim 5, wherein curing at least one of the first or the second deposits of adhesive includes illuminating at least one of the first or the second deposits of adhesive with ultraviolet light for a period of time.

12. A wirelessly detectable sponge, comprising:
a first sponge;
a first deposit of adhesive, the first deposit of adhesive which is deposited onto the first sponge at a first defined location;
a first wireless transponder, the first wireless transponder which is positioned on the first deposit of adhesive at a first defined location on the first sponge; and
a second deposit of adhesive, the second deposit of adhesive which is deposited to cover the first wireless transponder.

13. The wirelessly detectable sponge of claim 12, wherein the first sponge has a length, a width and a thickness, and wherein the first wireless transponder is positioned onto the first deposit of adhesive at the first defined location in a defined orientation relative to the length and width of the first sponge.

14. The wirelessly detectable sponge of claim 12, wherein the first wireless transponder is enclosed by a combination of the first deposit of adhesive and the second deposit of adhesive.

15. The wirelessly detectable sponge of claim 12, wherein the first deposit of adhesive includes a first amount of a first type of adhesive, and wherein the second deposit of adhesive includes a second amount of the first type of adhesive.

16. The wirelessly detectable sponge of claim 12, wherein a portion of the first sponge is folded to be between the first transponder and the second deposit of adhesive to cover the wireless transponder.

17. An assembly system that produces wirelessly detectable sponges, the assembly system comprising:
an assembly surface, the assembly surface which supports a first sponge;
a robotic appendage, the robotic appendage which picks a first wireless transponder from a supply of wireless transponders, and places the first wireless transponder onto the first sponge at a defined location;
at least one automated dispenser, the at least one automated dispenser which dispenses a first deposit of adhesive onto the first sponge at the defined location during a first time period before the first wireless transponder is placed onto the first sponge, and the at least one automated dispenser which dispenses a second deposit of adhesive onto the first sponge at the defined location during a second time period after the first wireless transponder is placed onto the sponge; and
an illumination source, the illumination source which is positioned to illuminate at least one of the first deposit of adhesive and the second deposit of adhesive with ultraviolet light.

18. The assembly system of claim 17, wherein the assembly surface is comprised of a conveyor.

19. The assembly system of claim 17, wherein the at least one automated dispenser includes a first automated dispenser that dispenses the first deposit of adhesive during the first time period and that dispenses the second deposit of adhesive during the second time period.

20. The assembly system of claim 17, wherein the at least one automated dispenser includes a first automated dispenser that dispenses the first deposit of adhesive during the first time period and a second automated dispenser that dispenses the second deposit of adhesive during the second time period.
